(19) United States
(12) Patent Application Publication
(10) Pub. No.: US 2007/0282216 A1
(43) Pub. Date: Dec. 6, 2007

(54) ALTERING BRAIN ACTIVITY THROUGH BINAURAL BEATS

(76) Inventors: Michael A. Vesely, Santa Cruz, CA (US); Nancy L. Clemens, Santa Cruz, CA (US)

Correspondence Address: FISH & RICHARDSON P.C., PO BOX 1022, MINNEAPOLIS, MN, 55440-1022, US

(21) Appl. No.: 11/756,561

(22) Filed: May 31, 2007

Related U.S. Application Data
(63) Continuation-in-part of application No. 11/292,376, filed on Nov. 28, 2005.
(60) Provisional application No. 60/632,085, filed on Nov. 30, 2004.

(51) Int. Cl. A61B 5/04

(52) U.S. Cl. 600/545

(57) ABSTRACT

The present disclosure includes, among other things, systems, methods and program products for brain balancing by inducing a binaural beat.

### CROSS-REFERENCE TO RELATED APPLICATIONS

This application is a continuation-in-part application of, and claims priority to, U.S. patent application Ser. No. 11/292,376, entitled Brain Balancing by Binaural Beat, to Vesely, et al., filed on Nov. 28, 2005, which claims priority to U.S. Provisional Application No. 60/632,085, entitled Brain Balancing by Binaural Beat, to Vesely, et al., which was filed on Nov. 30, 2004. The disclosures of both of the above applications are incorporated herein by reference in their entirety.

### BACKGROUND

The living brain exhibits electrical activity that varies in strength and frequency over time and from one area of the brain to another. An electroencephalogram (EEG) is useful in non-invasively observing human brain activity. An EEG is a recording of electrical signals from the brain made by attaching electrodes to a subject's scalp. These electrodes pick up electric signals naturally produced by the brain and send them to galvanometers (e.g., ammeters) that are in turn connected electronics, such as computers, to store the signals.

EEGs allow researchers to follow electrical impulses across the surface of the brain and observe changes over split seconds of time. An EEG can show what state a person is in—asleep, awake, anaesthetized—because the characteristic patterns of current differ for each of these states. One important use of EEGs has been to show how long it takes the brain to process various stimuli. Four general categories of continuous rhythmic sinusoidal EEG activity are typically recognized: Alpha, Beta, Delta and Theta. These are summarized in TABLE 1 below.

TABLE 1

| | APPROXIMATE | |
|---|---|---|
| TYPE OF | FREQUENCY | |
| RHYTHM | RANGE | DESCRIPTION |
| Beta | >13 Hz | Normal waking consciousness. Person |
| | | may be alert, aroused, concentrating, |
| | | active, busy, or anxious in this state. |
| Alpha | 8-13 Hz | Characteristic of a relaxed, alert |
| | | state of consciousness. Common in |
| | | meditative states and the “relaxa- |
| | | tion response” of the body. |
| Theta | 4-8 Hz | Typically found in adolescents with |
| | | learning disorders; also associated |
| | | with drowsiness. Present in |
| | | REM/dreaming sleep, and deep states of |
| | | meditation. |
| Delta | 0.5-4 Hz | The dominant rhythm in infants up to |
| | | one year and in stages three and four |
| | | of sleep (i.e., deep dreamless sleep.) |

In the last few years of EEG research, researchers have identified and created a new category of EEG frequency, called Gamma, which is generally regarded to be above 36 Hz. Also, Beta is commonly parsed into three separate categories: low, mid, and high Beta. For clarity of discussion, however, Beta will be treated as a single category. Furthermore, the implementations and techniques described in the present disclosure are able to use fewer or more frequency categories than those described in TABLE 1.

A so-called “binaural beat” frequency can be produced inside of the brain by supplying signals of different frequencies to the two ears of a subject. The binaural beat phenomenon was discovered in 1839 by H. W. Dove, a German experimenter. In general, when a subject receives signals of two different frequencies, one signal to each ear, the subject's brain detects a phase difference or other differences between these signals. When these signals are naturally occurring, the detected phased difference provides directional information to the higher centers of the brain. However, if these signals are provided through speakers or stereo earphones, the phase difference is detected as an anomaly. The resulting imposition of a consistent phase difference between the incoming signals causes the binaural beat in an amplitude modulated standing wave, within each superior olivary nucleus (sound processing center) of the brain. It is not possible to generate a binaural beat through an electronically mixed signal; rather, the action of both ears is required for detection of this beat.

Binaural beats result from the interaction of two different auditory impulses, originating in opposite ears, below 1000 Hz and which differ in frequency between 1-30 Hz. For example, if a pure tone of 400 Hz is presented to the right ear and a pure tone of 410 Hz is presented simultaneously to the left ear, an amplitude modulated standing wave of 10 Hz, the difference between the two tones, is experienced as the two wave forms mesh in and out of phase within the superior olivary nuclei.

In a sense, binaural beats are similar to beat frequency oscillations produced by a heterodyne effect, but occurring within the brain itself. If a binaural beat is within the range of a brain rhythm (e.g., Alpha, Beta, Theta, Delta), generally less than 30 Hz, the binaural beat can become an entrainment environment. The binaural beat is perceived as an auditory beat and theoretically can be used to entrain specific neural rhythms through a frequency-following response (FFR)—the tendency for cortical potentials to entrain to or resonate at the frequency of an external stimulus. In other words, if the brain is operating at one frequency, binaural beats of a fixed frequency can be produced within the brain so as to entice the brain to change its frequency to that of the binaural beat and thereby change the brain state. This effect has been used to study states of consciousness, to improve therapeutic intervention techniques, and to enhance educational environments.

As brain activity slows from beta to alpha to theta to delta, typically there is a corresponding increase in balance between the two hemispheres of the brain. This balanced brain state is called brain synchrony, or brain synchronization. Normally, brain rhythms exhibit asymmetrical patterns with one hemisphere dominant over the other. However, the balanced brain state offers deep tranquility, flashes of creative insight, euphoria, intensely focus attention, and enhanced learning abilities.

### SUMMARY

In general, one aspect of the subject matter described in this specification can be embodied in a method that includes obtaining a first electromagnetic emission measurement from a left hemisphere of a user's brain and a second electromagnetic emission measurement from a right hemisphere of the user's brain. An imbalance between the first and second measured emissions is detected based on the measurements, the imbalance indicative of a frequency imbalance between the left hemisphere and the right hemisphere. A binaural beat frequency is selected based on the frequency imbalance. A first audio signal is delivered to the user's left ear and a different second audio signal is delivered to the user's right ear to induce a binaural beat corresponding to the binaural beat frequency in the user. Other implementations of this aspect include corresponding systems, apparatus, and computer program products.

These and other implementations can optionally include one or more of the following features. Delivering includes during a period of time obtaining one or more additional electromagnetic emission measurements from the left and right hemispheres of the user's brain, and ceasing delivery of the first and second audio signals if an imbalance is no longer detected based on the additional measurements. Delivering can also include during a period of time obtaining one or more additional electromagnetic emission measurements from the left and right hemispheres of the user's brain, and modifying the binaural beat frequency during the period of time based on the additional measurements. The modifying includes one or more of changing the binaural beat frequency from being continuous to intermittent, or vice versa; introducing a time delay into the binaural beat frequency; introducing a phase delay into the binaural beat frequency; or changing the binaural beat frequency to a rest frequency.

These and other implementations can optionally include one or more of the following additional features. The imbalance is for a predominant frequency exhibited in the left and right hemispheres, the method further comprising moving the binaural beat frequency toward the predominant frequency over time. The imbalance is for a predominant frequency exhibited in the left and right hemispheres and where the binaural beat frequency is the predominant frequency. The binaural beat frequency is initially lower or higher than the predominant frequency. Delivering is maintained for a period of time. The delivering includes pausing the first and second audio signals for a duration corresponding to a rest period. The first audio signal and the second audio signal differ in magnitude, phase or both. The first audio signal and the second audio signal are in the range of 0.1 Hz to 40 Hz or 40 Hz to 400 Hz. The selecting includes selecting a binaural beat frequency based on a desired brain rhythm, and ceasing delivering of the first and second audio signals when the measurements indicate that the user's brain is exhibiting the desired brain rhythm.

Particular implementations of the subject matter described in this specification can be implemented to realize one or more of the following advantages. Users can automatically balance their brain rhythms and entrain their brain rhythms to a desired rhythm. Balancing and entrainment utilize electromagnetic feedback from the user's brain to guide the respective processes. Binaural beats are automatically induced in users in order to achieve entrainment or balancing. A user interface is provided which allows users to select parameters for balancing and entrainment, monitor their progress toward achieving these goals, and view their current brain state.

### BRIEF DESCRIPTION OF THE DRAWINGS

FIG. 1 is a schematic diagram of a system configured to automatically induce binaural beats in users.

FIG. 2A is a flowchart illustrating a technique for brain rhythm balancing using binaural beats.

FIG. 2B is a flowchart illustrating a further technique for brain rhythm balancing.

FIG. 3 is an example user interface for the system of FIG. 1.

FIG. 4. is a schematic diagram of a generic computer system.

Like reference numbers and designations in the various drawings indicate like elements.

### DETAILED DESCRIPTION

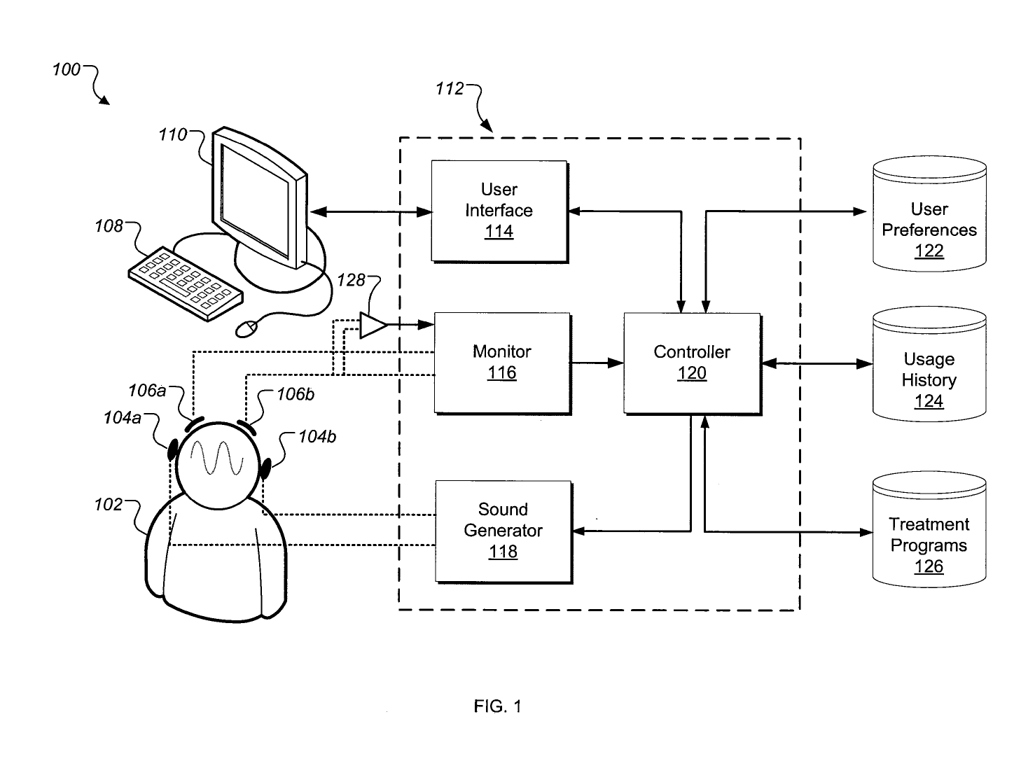

FIG. 1 is a schematic diagram of a system 100 configured to automatically induce binaural beats in users. A user 102 is equipped with two or more electromagnetic measurement devices (e.g., electrodes 106a-b) for measuring the user 102's brain electromagnetic activity. In various implementations, at least one measurement device (e.g., 106a) measures electromagnetic activity from the left hemisphere of the user 102's brain, and at least one measurement device (e.g., 106b) measures electromagnetic activity from the right hemisphere of the user 102's brain. The measurement devices are individually placed on or near the user 102's scalp, usually with a conductive gel. In some implementations, the measurement devices are placed in locations specified by the International 10-20 system. Alternatively, the measurement devices are integrated into an accessory such as eye glasses or headphones so that when the accessory is worn, the measurement devices are placed on or near the user 102's scalp. By way of illustration, measurement devices can be integrated into sides of eye glass frames, earphone covers, or other earphone parts.

In various implementations, the measurement devices 106a-b are connected to one or more amplifiers (e.g., differential amplifier 128). However, other arrangements of measurement devices and amplifiers are possible. Each amplifier produces a frequency (e.g., in Hz) that represents the difference between its inputs, possibly multiplied by a constant factor. The amplifier can be realized as a hardware component or a software component (e.g., monitor 116). By way of illustration, if electrode 106a measured a frequency of 8.4 Hz (Alpha rhythm) and electrode 106b measured a frequency of 9 Hz (Alpha rhythm), the amplifier 128 would produce a frequency equal to 0.6 Hz multiplied by a constant. This is referred to as the frequency imbalance. If both electrodes 106a-b measured the same frequency, the output of the amplifier 128 would be zero. If the two electrodes are measuring activity from different hemispheres of the user 102's brain, the amplifier 128 output indicates if the predominant frequency or rhythm (e.g., Alpha, Beta, etc.) is in a balanced or imbalanced state. In further implementations, a balanced state is an amplifier output from 0 Hz-T Hz and an imbalanced state is an amplifier output is greater than T Hz. The value of T can be determined based on a number of factors including the age of the user 102, medical conditions of the user 102, the predominate rhythm, and other factors.

The monitor component 116 receives digital or analog signals from the measurement devices 106a-b and, optionally, the amplifier 128. In some implementations, the signals are processed before being received by the monitor component 116 to remove artifacts or noise, or to perform other processing. The connection between the measurement devices 106a-b and the amplifier 128, and between the amplifier 128 and the monitor component 116 can be wired or wireless. The monitor component 116 determines the predominate rhythm based on the signals from the measurement devices. There are a number of ways the predominate rhythm can be determined. One approach is simply to average the frequencies measured by the measurement devices and identify which rhythm frequency range the average falls in. For instance, if electrode 106a measured 14.5 Hz and electrode 106b measured 16 Hz, the predominate rhythm would be Beta. Another approach is to use a weighted average of the frequencies where weights are assigned based on which region of the user 102's brain a given measurement device is measuring. Other approaches are possible. Using the received signals, the monitor component 116 can determine whether the predominate rhythm is in a balanced or imbalanced state in regards to the user 102's brain hemispheres. The predominate rhythm and an indication of the degree of imbalance are provided to the controller component 120.

The system 100 includes one or more computing devices 112 for execution of various software components, such as the monitor 116 and controller 120 components. Although several components are illustrated, there may be fewer or more components in the system 100. Moreover, the components can be distributed on one or more computing devices connected by one or more networks or other suitable communication means.

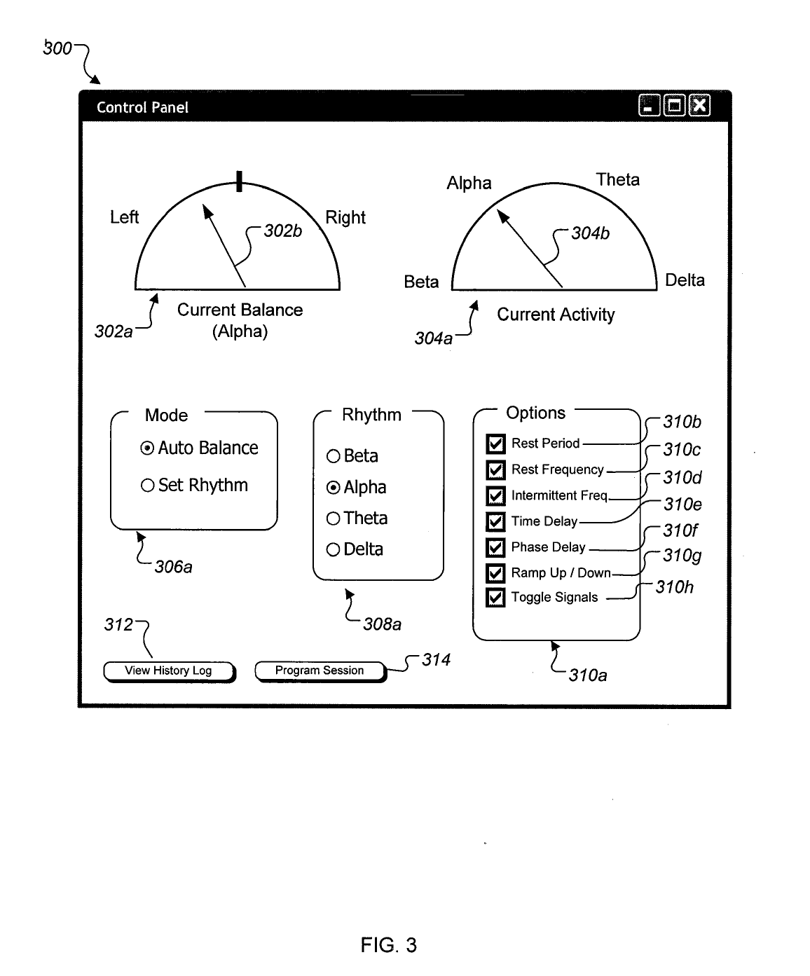

An optional user interface (UI) component 114 provides a graphical user interface (GUI) for the system 100. In various implementations, the UI 114 presents a graphical control panel 300 (FIG. 3) which allows users to monitor their current brain activity and provide settings to alter it. The UI 114 presents the control panel 300 on a display device 110 such as a liquid crystal display, for example. Users can interact with the control panel 300 using input devices 108 such as a keyboard, a computer mouse, video cameras (e.g., for gesture recognition), microphones (e.g., for voice and sound recognition), or other devices. The controller 120 provides information obtained from the monitor 116 to the UI 114 for display and accepts user settings from the UI 114 to control the sound generator component 118. The operation of the sound generator 118 will discussed in detail below. With reference to FIG. 3, an example control panel 300 provides information on the user's current brain state through meters 302a and 304a. Meter 304a displays the current predominant frequency for the user 102 as determined by the monitor component 1 16. For example, an animated needle 304b points to the current predominant frequency (e.g., Alpha) which can change over time. Meter 302a displays the current frequency imbalance for the predominant frequency. An animated needle 302b indicates the level of imbalance. For example, if the needle 302b is in a vertical orientation, the user 102's brain is in a balanced state. Otherwise, there is an imbalance in favor of the left or right hemispheres, which is indicated by the needle 302b pointing to the left or right side of the meter, respectively. The degree of the imbalance is indicated by the degree to which the needle 302b approaches a horizontal orientation.

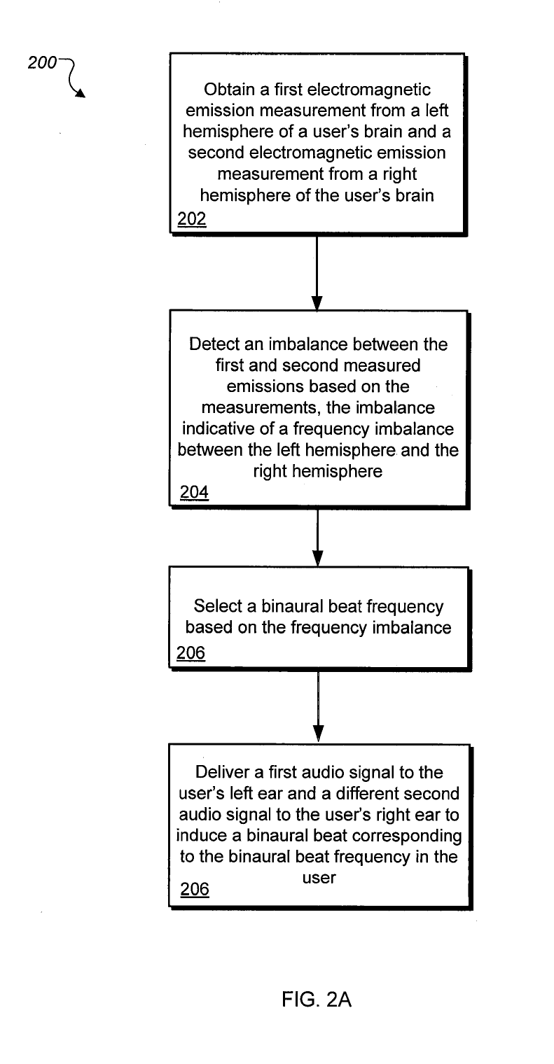

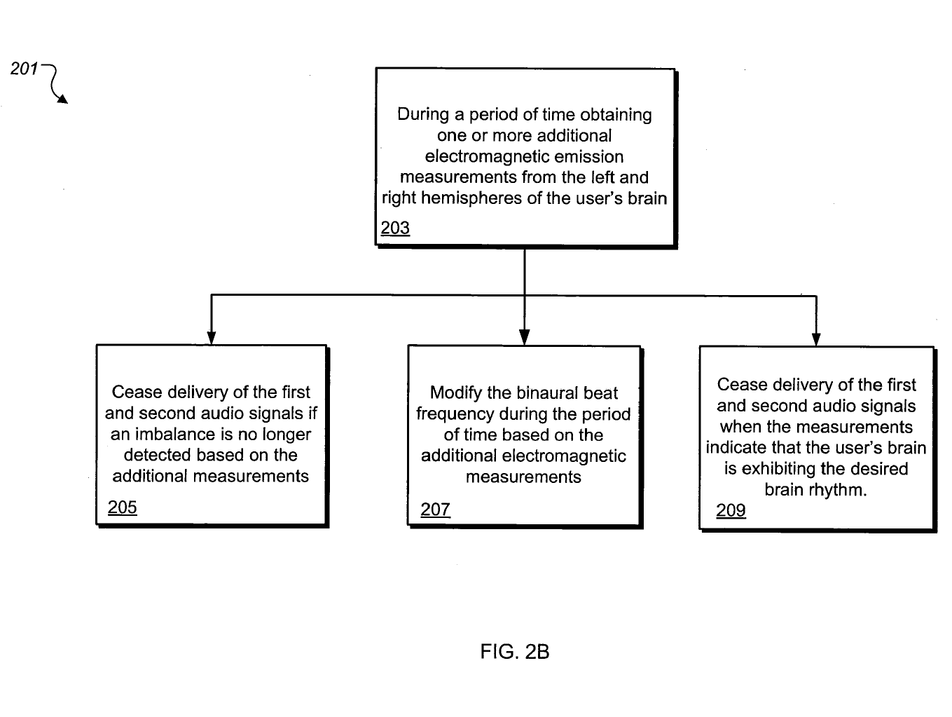

The control panel 300 also allows the user 102 to provide settings to the system 100 which determine how the system 100 provides binaural beat inducing sounds to the user 102 through the sound generator 118. In various implementations, the user can set an overall system mode to “auto balance” or “set rhythm”. In the auto balance mode, the system 100 will automatically balance the user 102's current predominant frequency if this frequency is not in a balanced state. This is further described with reference to FIG. 2A.

Selection of the set rhythm mode in the control panel 300 causes the controller 120 to follow a program to automatically bring the user 102's brain rhythm to a desired rhythm 308a, if the user 102's predominant frequency is not equal to the desired rhythm 308a. For example, the user 102 who desires to study efficiently may wish to put their brain in an Alpha rhythm state since Alpha rhythms are characteristic of an alert state of consciousness. This is further described with reference to FIG. 2B. In various implementations, the control panel 300 settings are saved in persistent storage 122 so that subsequent uses of the system 100 will not require the settings to be input again.

The sound generator 118 generates a binaural beat frequency selected by the controller based on the frequency imbalance, desired rhythm or both (step 206). An audio signal is then delivered to the user's left ear and a different audio signal is delivered to the user's right ear by the sound generator 118 and through headphones 104a-b to induce a binaural beat corresponding to the binaural beat frequency. The headphones can receive signals through wires or wirelessly from the sound generator 118. Generally, the binaural beat frequency that the brain can detect, ranges from approximately 0 to 100 Hz. The ear has the greatest sensitivity at around 1000 Hz. However, this frequency is not pleasant to listen to, and a frequency of 100 Hz is too low to provide a good modulation index. Thus, in some implementations the frequencies between 100 Hz and 1000 Hz are normally used for binaural beat, and preferably between 100 Hz and 400 Hz. Typically, the frequency of 200 Hz is a good compromise between sensitivity and pleasing sounds.

The audio signals can be produced in a number of ways. For example, the sound generator 118 can be used to produce the audio signals and listened to through headphones. Alternatively, analog operational amplifiers and other integrated circuitry can be provided in conjunction with a set of headphones to produce such audio signals. These signals may be recorded on a machine readable medium and played through a set of earphones. Headphones are necessary because otherwise the beat frequency would be produced in the air between the two speakers. This would produce audible beat notes, but would not produce the binaural beats within the brain.

FIG. 2A is a flowchart illustrating a technique 200 for brain rhythm balancing using binaural beats. Initially, a first electromagnetic emission measurement from a left hemisphere of the user 102's brain and a second electromagnetic emission measurement from a right hemisphere of the user 102's brain are obtained (e.g., from the measurement devices 106a-b; step 202). An imbalance is then detected between the first and second measured emissions, the imbalance indicative of a frequency imbalance between the left hemisphere and the right hemisphere of the user 102's brain (step 204). A binaural beat frequency is then selected (e.g., by the controller 120) based on the frequency imbalance (step 206). A first audio signal is then delivered to the user's left ear and a different second audio signal to the user's right ear (e.g., by the sound generator 118 and through headphones 104a-b) to induce a binaural beat in the user 102 corresponding to the binaural beat frequency in the user 102 (step 208).

In various implementations, the control panel 300 allows the user 102 to set various combinations of options 310a for how the binaural beat will be induced in the user 102. For example, the binaural beat can be continuous or intermittent 310d. The binaural beat can be maintained for some predetermined period of time, after which a new frequency can be determined. Another possibility would be to take the user 102 to a rest frequency between sessions 310c. Another possibility would be to allow the user 102 to rest between sessions, e.g. generating no signal at all for a period of time 310b. The binaural beat can start at the correcting or desired frequency, or can start at a higher or lower frequency and then moves toward the correcting or desired frequency 310g. The binaural beat can phase lock onto a certain brain wave frequency of the person and to gently carry down to the desired frequency. The scanning or continuously varying frequency can be important since the different halves generally operate at different brain frequencies. This is because one brain half is generally dominant over the other brain half. Therefore, by scanning at different frequencies from a higher frequency to a lower frequency, or vice versa, each brain half is locked onto the respective frequency and carried down or up so that both brain halves are operating synchronously with each other and are moved to the desired frequency brain wave pattern corresponding to the chosen state.

Another type is to raise the brain wave frequency, and particularly, to increase the performance of the person, for example, in sporting events. In this mode, both ears of the person are supplied with the same audio signal having a substantially continuously varying frequency which varies, for example, from 20 Hz to 40 Hz, although the signals are amplitude and/or phase modulated. It is believed that, if the brain wave frequency of the person is less than 20 Hz, the brain will phase lock onto audio signals of the same frequency or multiples of the same frequency. Thus, even if the brain is operating at a 10 Hz frequency rate, when an audio signal of 20 Hz is supplied, the brain will be phase locked onto such a signal and may be nudged up as the frequency is increased. Without such a variation in frequency of the audio signal, the brain wave frequency will phase lock thereto, but will not be nudged up. The audio signal can be changed from 20 Hz to 40 Hz in a time period of approximately 5 minutes and continuously repeats thereafter so as to nudge the brain frequency to a higher frequency during each cycle.

In various implementations, a constant frequency of 200 Hz audio signal can supplied to one ear (for example, the left ear) and another audio signal having a frequency which ranges from 300 Hz to 200 Hz is applied to the other ear (for example, the right ear). As a result, binaural beats at 0-100 Hz are produced in the brain. The audio signals can be toggled by user 102 selection of control 310h, meaning a constant frequency can be applied to the right ear and the varied frequency applied to the left ear. Further the toggle can happen at a fast rate. This toggle rate can help to maintain the attention span of the brain during the binaural beat generation and might allow the user to perceive the signal moving back and forth between the left and right ears. Further, the left and right ear signals can have different time delay 310e or phase differences 310f since, for low frequencies of this nature, the time delay or phase difference between the left and right signals could produce a greater effect than the relative amplitude to the brain. The time delay could be up to a few seconds and the phase difference can be anywhere from 0 to 360°.

In further implementations, additional options can be specified, the amplitude and waveform of the applied frequencies can be constant, selected by the user, or can vary. For example, if the user 102 selects the program session button 314 of the control panel 300, the user can interactively create a treatment program that varies signal properties and options over time. Treatment programs can be saved in persistent storage 126 and invoked when the user 102 wishes to run the program. In this case, the controller 120 can use the stored treatment program and, optionally, input from the monitor component 116 to guide the sound generator 118. In some implementations, the user 102's usage history is automatically recorded and stored in 124 for recall later by invoking button 312, for instance. The usage history for a given session or program is a recording of the input received by the controller 120, user options, and the output from the sound generator 118 over time. Saved usage histories can be stored as treatment programs 126.

FIG. 2B is a flowchart illustrating a further technique 201 for brain rhythm balancing. During a period of time obtaining one or more additional electromagnetic emission measurements from the left and right hemispheres of the user's brain are obtained (step 203). Delivery of the first and second audio signals is then ceased if an imbalance is no longer detected based on the additional measurements (step 205). Alternatively, the binaural beat frequency is modified during the period of time based on the additional measurements (step 207). In a further alternative, the delivering of the first and second audio signals are ceased when the measurements indicate that the user's brain is exhibiting the desired brain rhythm (step 209).

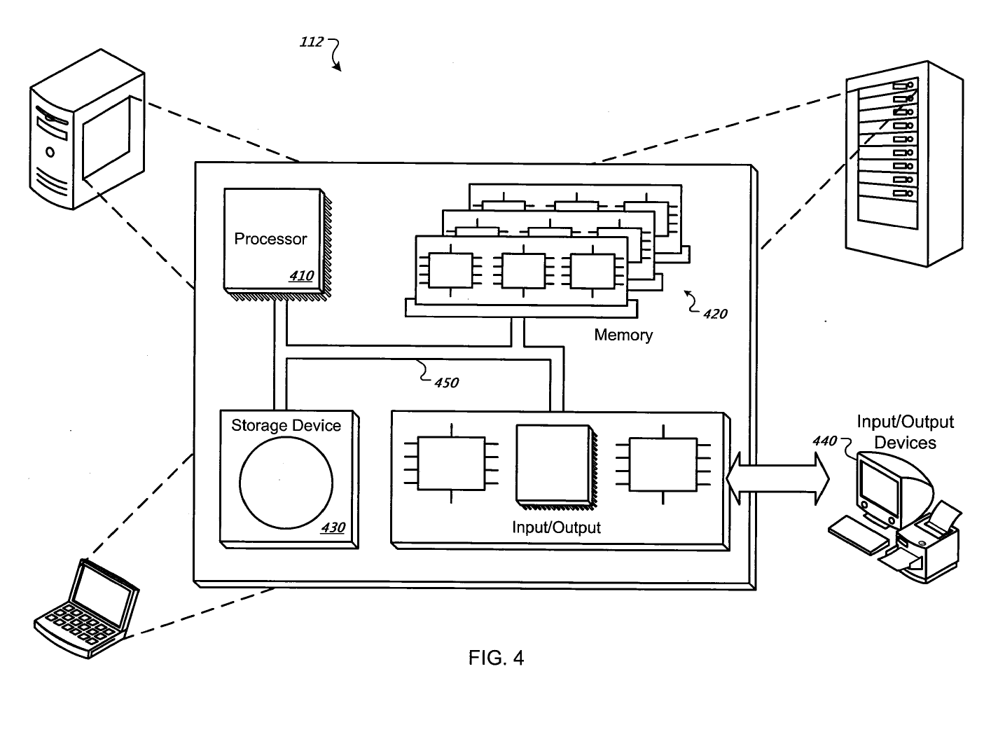

FIG. 4 is a schematic diagram of a generic computer device 112 which can be used in association with practice of the techniques 200 and 201, for example. The device 112 can be embodied in a personal computer, a work station, a portable computer, a digital media player (e.g., an Apple ipod), a mobile phone (e.g., a smart phone), a pillow, or an electronic game (e.g., a Sony Playstation Portable), for example. The device 112 can include a processor 410, a memory 420, a storage device 430, and input/output devices 440. Each of the components 410, 420, 430, and 440 are interconnected using a system bus 450. The processor 410 is capable of processing instructions for execution within the device 112. Such executed instructions can implement one or more components of system 100, for example. In one implementation, the processor 410 is single or multi-threaded and single or multi-core. The processor 410 is capable of processing instructions stored in the memory 420 or on the storage device 430 to display graphical information for a user interface on the input/output device 440.

The memory 420 is a computer readable medium such as volatile or non volatile random access memory that stores information within the device 112. The memory 420 could store user preferences 122, usage history 124 or treatment programs 126, or information required by the control panel 300, for example. The storage device 430 is capable of providing persistent storage for the device 112. The storage device 430 may be a floppy disk device, a hard disk device, an optical disk device, or a tape device, or other suitable persistent storage means. The input/output device 440 provides input/output operations for the device 112. In one implementation, the input/output device 440 includes a keyboard and/or pointing device. In another implementation, the input/output device 440 includes a display unit for displaying graphical user interfaces (e.g., 300).

Implementations of the subject matter and the functional operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a computer-readable medium for execution by, or to control the operation of, data processing apparatus. The computer-readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal, that is generated to encode information for transmission to suitable receiver apparatus.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described is this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular implementations of the invention. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the invention have been described. Other implementations are within the scope of the following claims. For example, the actions recited in the claims can be performed in a different order and still achieve desirable results.

What is claim is:

1. A computer-implemented method, comprising:
obtaining a first electromagnetic emission measurement from a left hemisphere of a user's brain and a second electromagnetic emission measurement from a right hemisphere of the user's brain;
detecting an imbalance between the first and second measured emissions based on the measurements, the imbalance indicative of a frequency imbalance between the left hemisphere and the right hemisphere;
selecting a binaural beat frequency based on the frequency imbalance; and
delivering a first audio signal to the user's left ear and a different second audio signal to the user's right ear to induce a binaural beat corresponding to the binaural beat frequency in the user.

2. The method of claim 1 where delivering includes:
during a period of time obtaining one or more additional electromagnetic emission measurements from the left and right hemispheres of the user's brain; and
ceasing delivery of the first and second audio signals if an imbalance is no longer detected based on the additional measurements.

3. The method of claim 1 where delivering includes:
during a period of time obtaining one or more additional electromagnetic emission measurements from the left and right hemispheres of the user's brain; and
modifying the binaural beat frequency during the period of time based on the additional measurements.

4. The method of claim 3 where the modifying includes one or more of:
changing the binaural beat frequency from being continuous to intermittent, or vice versa;
introducing a time delay into the binaural beat frequency;
introducing a phase delay into the binaural beat frequency; or
changing the binaural beat frequency to a rest frequency.

5. The method of claim 1 where the imbalance is for a predominant frequency exhibited in the left and right hemispheres, the method further comprising:
moving the binaural beat frequency toward the predominant frequency over time.

6. The method of claim 1 where the imbalance is for a predominant frequency exhibited in the left and right hemispheres and where the binaural beat frequency is the predominant frequency.

7. The method of claim 5 where the binaural beat frequency is initially lower or higher than the predominant frequency.

8. The method of claim 1 where delivering is maintained for a period of time.

9. The method of claim 1 where delivering includes:
pausing the first and second audio signals for a duration corresponding to a rest period.

10. The method of claim 1 where the first audio signal and the second audio signal differ in magnitude, phase or both.

11. The method of claim 1 where first audio signal and the second audio signal are in the range of 0.1 Hz to 40 Hz or 40 Hz to 400 Hz.

12. The method of claim 1 where the selecting includes:
selecting a binaural beat frequency based on a desired brain rhythm; and
ceasing delivering of the first and second audio signals when the measurements indicate that the user's brain is exhibiting the desired brain rhythm.

13. A computer program product, encoded on a computer-readable medium, operable to cause data processing apparatus to perform operations comprising:
obtaining a first electromagnetic emission measurement from a left hemisphere of a user's brain and a second electromagnetic emission measurement from a right hemisphere of the user's brain;
detecting an imbalance between the first and second measured emissions based on the measurements, the imbalance indicative of a frequency imbalance between the left hemisphere and the right hemisphere;
selecting a binaural beat frequency based on the frequency imbalance; and
delivering a first audio signal to the user's left ear and a different second audio signal to the user's right ear to induce a binaural beat corresponding to the binaural beat frequency in the user.

14. A system comprising:
an electromagnetic measurement device configured to measure electromagnetic emissions from a user's brain;
an audio generator configured to deliver differing audio signals to the user's ears to induce a binaural beat in the user's brain; and
one or more computing devices configured to perform operations comprising:
obtaining from the measurement device a first electromagnetic emission measurement from a left hemisphere of the user's brain and a second electromagnetic emission measurement from a right hemisphere of the user's brain;
detecting an imbalance between the first and second measured emissions based on the measurements, the imbalance indicative of a frequency imbalance between the left hemisphere and the right hemisphere;
selecting a binaural beat frequency based on the frequency imbalance; and
delivering using the audio generator a first audio signal to the user's left ear and a different second audio signal to the user's right ear to induce a binaural beat corresponding to the binaural beat frequency in the user.

15. The system of claim 14 where delivering includes performing further operations comprising:
during a period of time obtaining one or more additional electromagnetic emission measurements from the left and right hemispheres of the user's brain; and
ceasing delivery of the first and second audio signals if an imbalance is no longer detected based on the additional measurements.

16. The system of claim 14 where delivering includes performing further operations comprising:
during a period of time obtaining one or more additional electromagnetic emission measurements from the left and right hemispheres of the user's brain; and
modifying the binaural beat frequency during the period of time based on the additional measurements.

17. The method of claim 16 where the modifying includes performing further operations comprising one or more of:
changing the binaural beat frequency from being continuous to intermittent, or vice versa;
introducing a time delay into the binaural beat frequency;
introducing a phase delay into the binaural beat frequency; or
changing the binaural beat frequency to a rest frequency.

18. The system of claim 14 where the imbalance is for a predominant frequency exhibited in the left and right hemispheres, performing operations further comprising:
moving the binaural beat frequency toward the predominant frequency over time.

19. The system of claim 14 where the imbalance is for a predominant frequency exhibited in the left and right hemispheres and where the binaural beat frequency is the predominant frequency.

20. The method of claim 18 where the binaural beat frequency is initially lower or higher than the predominant frequency.

21. The system of claim 14 where delivering is maintained for a period of time.

22. The system of claim 14 where delivering includes performing operations further comprising:
pausing the first and second audio signals for a duration corresponding to a rest period.

23. The system of claim 14 where the first audio signal and the second audio signal differ in magnitude, phase or both.

24. The system of claim 14 where first audio signal and the second audio signal are in the range of 0.1 Hz to 40 Hz or 40 Hz to 400 Hz.

25. The system of claim 14 where the selecting includes performing operations further comprising:
selecting a binaural beat frequency based on a desired brain rhythm; and
ceasing delivering of the first and second audio signals when the measurements indicate that the user's brain is exhibiting the desired brain rhythm.

26. The system of claim 14, further comprising:
a user interface configured to control the selecting or present information based on the obtained measurements.
